(19) United States
(12) Patent Application Publication
(10) Pub. No.: US 2026/0224171 A1
(43) Pub. Date: Aug. 6, 2026

(54) APPARATUS AND METHOD FOR PREDICTING INTRADIALYTIC HYPOTENSION IN PATIENT

(71) Applicant: Electronics and Telecommunications Research Institute, Daejeon (KR)

(72) Inventors: Tae Wuk BAE, Daejeon (KR); Kee Koo KWON, Daejeon (KR); Kwang Yong KIM, Daejeon (KR); Woo Jin BYUN, Daejeon (KR)

(73) Assignee: Electronics and Telecommunications Research Institute, Daejeon (KR)

(21) Appl. No.: 19/229,323

(22) Filed: Jun. 5, 2025

(30) Foreign Application Priority Data
Feb. 5, 2025 (KR) 10-2025-0014500

(51) Int. Cl. A61B 5/00; A61B 5/024

(52) U.S. Cl. CPC A61B 5/7275; A61B 5/02405

(57) ABSTRACT

The present invention relates to an apparatus and method for predicting intradialytic hypotension in a patient, and the apparatus for predicting intradialytic hypotension in a patient includes a measurement module configured to measure a heart rate of a patient, a database, and a processor operatively coupled to the measurement module and the database, wherein the processor calculates an average heart rate and a heart rate fitting curve of heart rate signals of the patient measured by the measurement module for a set time, analyzes a change in heart rate based on the average heart rate and the heart rate fitting curve, and predicts occurrence of intradialytic hypotension.

### CROSS-REFERENCE TO RELATED APPLICATION

The present application claims priority and the benefit of Korean Patent Application No. 10-2025-0014500, filed on February 5, 2025 in the Korean Intellectual Property Office, the entire disclosure of which is incorporated herein by reference.

### BACKGROUND

### 1. Field of the Invention

The present invention relates to an apparatus and method for predicting intradialytic hypotension in a patient using a change in heart rate of the patient.

### 2. Discussion of Related Art

Intradialytic hypotension (IDH) is the most common complication in hemodialysis patients and is a serious complication associated with an increased mortality in addition to an increased hospitalization period, an increased mortality, cerebral ischemia, vascular access thrombosis, and cardiovascular disease.

Since IDH is defined in various ways, an incidence percentage of the IDH has also been reported differently, but it is known that the IDH is caused by various factors such as dialysis-related factors such as temperature of a dialysis fluid, a composition of a dialysis fluid electrolyte, a ultrafiltration rate, etc., administered drugs, heart and blood vessel conditions, and an autonomic nervous system function in addition to an old age, a female sex, a long dialysis period, diabetes, arteriosclerosis, left ventricular hypertrophy, predialysis diet, a high body mass index, a low albumin level, etc.

The IDH, which is a rapid decrease in blood pressure during hemodialysis, is a major risk to dialysis patients, is reported as occurring in about 15 to 20% of the dialysis patients, and complicates about 15% to 30% of all hemodialysis treatments.

The IDH means a decrease of 10 mmHg or more in mean arterial pressure or a decrease of 20 mmHg or more in systolic blood pressure.

The IDH can cause various risks to a central nervous system, a gastrointestinal system, heart, and kidneys, and frequent occurrence of the IDH is an important risk factor that increases the mortality percentage of dialysis patients. In addition, the IDH can cause serious arrhythmia, which can greatly affect sudden cardiac death.

Accordingly, a technology to predict IDH is very important for hemodialysis patients, but the corresponding technology does not sufficiently developed yet. In addition, in the case of past studies, prediction had been made only using static data such as basic information of a patient, underlying disease, and in-body chemical compounds during dialysis, and thus it was impossible to accurately predict whether IDH that requires real-time performance occurred.

The background technology of the present invention is disclosed in Korean Laid-Open Patent No. 10-2020-0144312 (published on December 29, 2020).

The above information disclosed in this Background section is for enhancement of understanding of the background of the present invention, and therefore, it may contain information that does not constitute the related art.

### SUMMARY OF THE INVENTION

The present invention is directed to providing an apparatus and method for predicting intradialytic hypotension in a patient, which predict the occurrence of intradialytic hypotension using a heart rate fitting curve and an average heart rate of a heart rate signal of a dialysis patient.

However, objects of the present invention are not limited to the above objects, and other objects that are not specifically mentioned herein will be clearly understood by those skilled in the art based on the description of the present invention below.

According to an aspect of the present invention, there is provided an apparatus for predicting intradialytic hypotension in a patient includes a measurement module configured to measure a heart rate of a patient, a database, and a processor operatively coupled to the measurement module and the database, wherein the processor calculates an average heart rate and a heart rate fitting curve of heart rate signals of the patient measured by the measurement module for a set time, analyzes a change in heart rate based on the average heart rate and the heart rate fitting curve, and predicts occurrence of intradialytic hypotension.

The processor calculates the heart rate fitting curve by fitting a multi-order polynomial on the heart rate signals.

The processor analyzes the change in heart rate based on the average heart rate and the heart rate fitting curve for the set time at a set cycle interval.

The processor predicts the occurrence of intradialytic hypotension when at least one of a percentage of a maximum area among areas of the average heart rate or higher and a percentage of a maximum area among areas of less than the average heart rate in the heart rate fitting curve for the set time at a set cycle interval is a set value or more.

The processor generates an alarm when a sum of heart rates of the average heart rate or higher or a sum of heart rates of less than the average heart rate at each set cycle exceeds a set maximum value.

According to another aspect of the present invention, there is provided a method of predicting intradialytic hypotension in a patient includes receiving, by a processor, heart rate signals of a patient measured by a measurement module, calculating, by the processor, an average heart rate and a heart rate fitting curve of the heart rate signals for a set time, and analyzing, by the processor, a change in heart rate based on the average heart rate and the heart rate fitting curve and predicting occurrence of intradialytic hypotension.

The calculating of the heart rate fitting curve includes calculating, by the processor, the heart rate fitting curve by fitting a multi-order polynomial on the heart rate signals.

The analyzing of the change in heart rate and predicting of the occurrence of intradialytic hypotension includes analyzing, by the processor, the change in heart rate based on the average heart rate and the heart rate fitting curve for the set time at a set cycle interval.

The analyzing of the change in heart rate and predicting of the occurrence of intradialytic hypotension includes predicting, by the processor, the occurrence of intradialytic hypotension when at least one of a percentage of a maximum area among areas of the average heart rate or higher and a percentage of a maximum area among areas of less than the average heart rate in the heart rate fitting curve for the set time at a set cycle interval is a set value or more.

The analyzing of the change in heart rate and predicting of the occurrence of intradialytic hypotension includes generating, by the processor, an alarm when a sum of heart rates of the average heart rate or higher or a sum of heart rates of less than the average heart rate at each set cycle exceeds a set maximum value.

### BRIEF DESCRIPTION OF THE DRAWINGS

The following drawings attached to the present specification illustrate embodiments of the present invention and further describe aspects and features of the present invention together with the detailed description of the present invention. Thus, the present invention should not be construed as being limited to the drawings, in which:

FIG. 1 is a block diagram showing an apparatus for predicting intradialytic hypotension (IDH) in a patient according to one embodiment of the present invention;

FIG. 2 is a graph showing changes in heart rate of an IDH patient and a normal patient in the apparatus for predicting IDH according to one embodiment of the present invention;

FIG. 3 is an exemplary view showing an IDH indicator according to changes in heart rate in the apparatus for predicting IDH in patients according to one embodiment of the present invention;

FIG. 4 is an exemplary view showing an IDH indicator of a hypotension patient by each time section during a set time in the apparatus for predicting IDH according to one embodiment of the present invention; and

FIG. 5 is a flowchart for describing a method of predicting IDH in a patient according to one embodiment of the present invention.

### DETAILED DESCRIPTION OF EXEMPLARY EMBODIMENTS

Hereinafter, an apparatus and method for predicting intradialytic hypotension (IDH) in a patient according to one embodiment of the present invention will be described.

In this description, thicknesses of lines, sizes of components, etc., shown in the drawings may be exaggerated for clarity and convenience of the description. In addition, terms to be described below are the terms defined in consideration of functions in the present invention, which may be changed according to a user or operator’s intention or custom. Accordingly, the definition of these terms should be made based on the contents throughout the specification.

Hereinafter, embodiments of the present invention will be described in detail with reference to the accompanying drawings so that those skilled in the art to which the present invention pertains can easily carry out the present invention. However, the present invention may be implemented in various different forms and is not limited to the embodiments described herein. In addition, to clearly describe the present invention in the drawings, parts irrelevant to the description have been omitted, and throughout the specification, similar parts have been denoted as similar reference numerals.

Throughout the specification, when a certain portion is described as “including” a certain element, it means that the certain portion may further include another element rather than precluding another element unless specifically stated to the contrary.

The implementation described in the present specification may be implemented, for example, as a method or process, device, a software program, a data stream, or a signal. Although described only in the context of the implementation of a single form (e.g., only a method is described), the implementations of the described features may also be implemented in other forms (e.g., devices or programs). The device may be implemented with appropriate hardware, software, firmware, etc. The method may be implemented by a device such as a processor, which is generally referred to as a processing device including, for example, a computer, a microprocessor, an integrated circuit, a programmable logic device, etc.

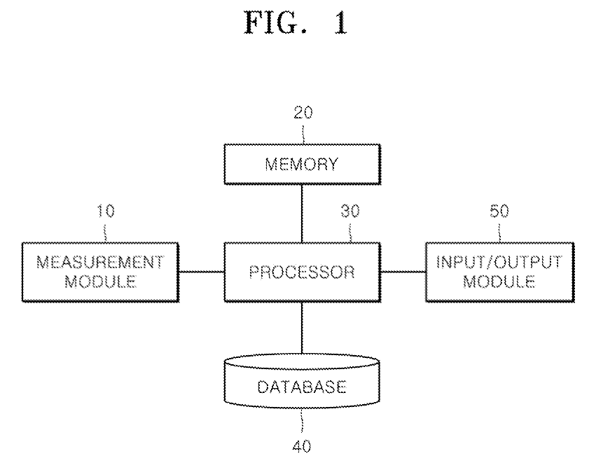

FIG. 1 is a block diagram showing an apparatus for predicting IDH in a patient according to one embodiment of the present invention.

As shown in FIG. 1, the apparatus for predicting IDH in a patient according to one embodiment of the present invention may include a measurement module 10, a database 40, an input/output module 50, a memory 20, and a processor 30.

The measurement module 10 may measure a heart rate of a patient during dialysis and provide a heart rate signal. Here, the measurement module 10 may include an electrocardiograph and measure and provide a heart rate at 1-second interval.

The database 40 may store the heart rate signal of the patient measured by the measurement module 10.

In the present embodiment, the database 40 may store 40-minute-long heart rate signals measured at 1-second interval.

The input/output module 50 may be connected to a network as a unit for a user interface and may input setting values or commands for the operation of the apparatus for predicting IDH in a patient.

For example, the input/output module 50 may include a device, such as a microphone, a keyboard, a mouse, etc., for input and a device such as a display or a speaker for output. As another example, the input/output module 50 may include a device that integrates functions for input and output, such as a touch screen. In addition, the input/output module 50 may be configured as a single device, such as a computer device.

The memory 20 may store an execution program and related data for the apparatus for predicting IDH in a patient, and the stored pieces of information may be selected by the processor 30 as needed.

That is, various types of data and commands generated during the execution process of an operating system (O/S) or an application (a program or an applet) for driving the apparatus for predicting IDH in a patient are stored in the memory 20. In this case, the memory 20 may be implemented as a nonvolatile memory, a volatile memory, a flash memory, a hard disk drive, a solid state drive (SSD), etc. In addition, the memory 20 may be accessed, and data may be read/recorded/modified/deleted/updated by the processor 30.

The processor 30 may be operatively coupled to the measurement module 10, the database 40, the input/output module 50, and the memory 20 and may copy various programs stored in the memory 20 to a random access memory (RAM) and execute the programs to perform various operations in order to control the overall operation of the apparatus for predicting IDH in a patient.

Here, the processor 30 is described as including only one central processing unit (CPU) but may be implemented as a plurality of CPUs (or digital signal processors (DSPs), system on chip (SoCs), etc.) when implemented.

In various embodiments, the processor 30 may be implemented as a DSP, a microprocessor, or a time controller (TCON) that processes digital signals. However, the processor 30 is not limited thereto, and may include one or more of a CPU, a micro controller unit (MCU), a micro processing unit (MPU), a controller, an application processor (AP), a communication processor (CP), or an ARM processor or may be defined as the corresponding term. In addition, the processor 30 may be implemented as an SoC or a large scale integration (LSI) with a built-in processing algorithm or may be implemented in the form of a field programmable gate array (FPGA).

That is, the processor 30 executes the execution program stored in the memory 20 and then calculates an average heart rate and a heart rate fitting curve of the heart rate signals of the patient measured for a set time from the measurement module 10.

In the present embodiment, the processor 30 may calculate the average heart rate with respect to the heart rate signal of the patient measured for 40 minutes and calculate a heart rate fitting curve fitted with a 20^{th}-order polynomial.

Here, since the heart rate signal is rough to analyze, a fitting curve that approximates data with the heart rate fitting curve may be generated to identify the overall trend of the heart rate.

After calculating the average heart rate and the heart rate fitting curve in this way, the processor 30 may analyze a change in the heart rate based on the average heart rate and the heart rate fitting curve and predict the occurrence of IDH.

The processor 30 may analyze the change in heart rate based on the average heart rate and the heart rate fitting curve for the set time at a set cycle interval.

In the present embodiment, the processor 30 may analyze the change in heart rate based on heart rate signals for 40 minutes at a 5-minute cycle interval.

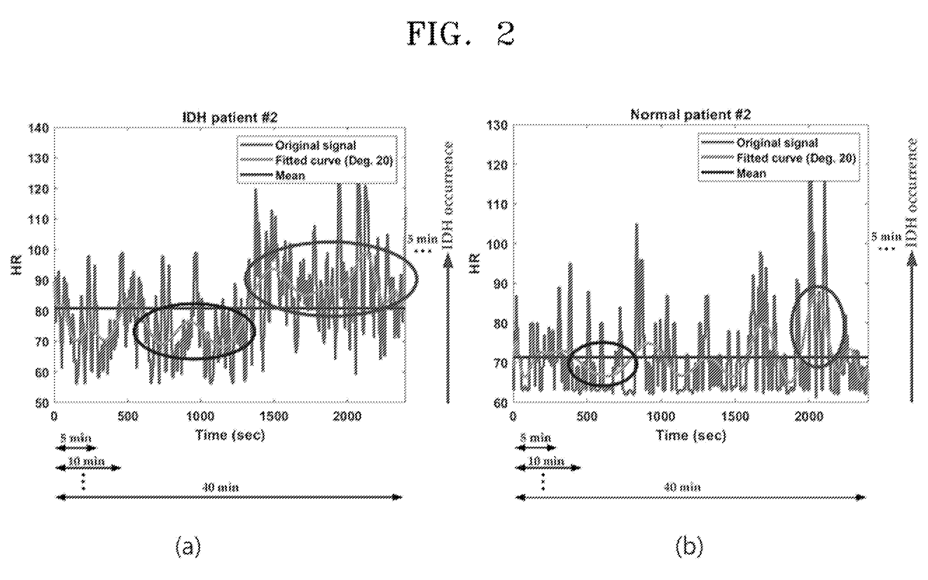

FIG. 2 is a graph showing changes in heart rate of IDH in a patient and a normal patient in the apparatus for predicting IDH according to one embodiment of the present invention.

First, based on the graph showing changes in heart rate of IDH in the patient and the normal patient as shown in FIG. 2, the characteristics of the heart rate signal for predicting IDH are as follows.

To analyze the characteristics of the heart rate signal of a dialysis patient before the occurrence of IDH, 40-minute-long heart rate signals up to a time point of 5 minutes before the occurrence of IDH may be checked.

That is, by adding a signal at 5-minute interval (300 points, 1 point/1 second) from a time point of 45 minutes before the occurrence of IDH, heart rate analysis may be performed for a total of 8 time sections from 5-minute-long heart rate signals to 40-minute-long heart rate signals.

FIG. 2A shows a heart rate signal of an IDH patient and FIG. 2B shows a heart rate for a normal patient, in which a purple horizontal line indicates an average heart rate. In addition, a green heart rate fitting curve is a curve fitted with a 20^{th}-order polynomial with respect to the heart rate signal.

Here, based on the average heart rate, a higher change in heart rate (a red circle) may indirectly indicate an increased blood pressure, while a lower change in heart rate (a blue circle) may indicate a decreased blood pressure.

When reviewing the change in heart rate, the phenomenon of a higher heart rate after a lower heart rate can be seen as the blood pressure compensatory mechanism of the body for raising the decreased blood pressure.

Accordingly, the change in heart rate may be analyzed through an area of a space generated by the average heart rate line and the heart rate fitting curve. That is, a total of heart rates in the maximum area generated at more than the average heart rate and the maximum area generated at less than the average heart rate may be used as a value representing the change in heart rate in a corresponding time section.

Accordingly, the processor 30 may predict the occurrence of IDH when at least one of a percentage of the maximum area among areas of the average heart rate or higher in the heart rate fitting curve and a percentage of the maximum area of areas of less than the average heart rate in the heart rate fitting curve for the set time at the set cycle interval is a set value or more.

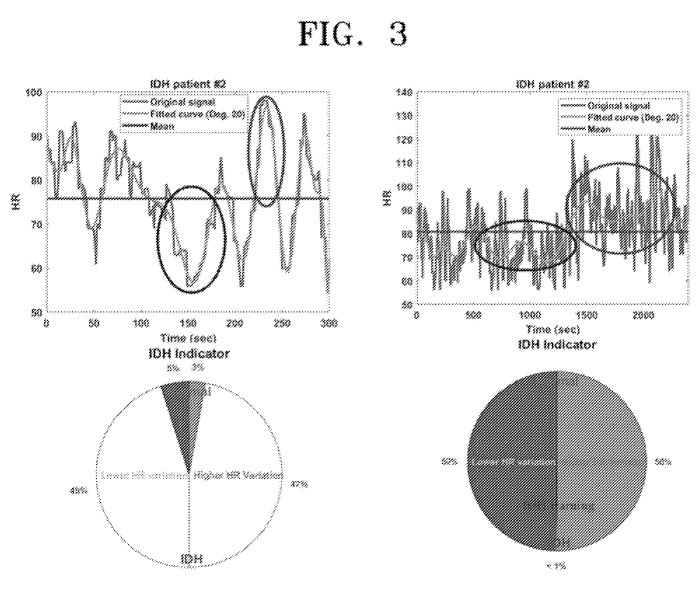

FIG. 3 is an exemplary view showing an IDH indicator according to changes in heart rate in the apparatus for predicting IDH in patients according to one embodiment of the present invention.

As shown in FIG. 3, heart rate signals corresponding to initial 5 minutes (300 points) and the remaining 40 minutes (2,400 points) up to a time point of 5 minutes before the occurrence of IDH and a corresponding IDH indicator can be shown.

Specifically, in each heart rate signal, based on the average heart rate, the blue circle represents an area corresponding to the maximum sum of high heart rates, and the red circle represents an area corresponding to the maximum sum of low heart rates.

In this case, left and right sides of the IDH indicator that indicates a change in heart rate represent rates of the maximum sums of the low heart rates and the high heart rates, respectively, which can indirectly show incidence rates of low and high blood pressures.

That is, in the initial 5-minute (300 point) section, the percentage of the maximum sum (area) of the low heart rates is 5%, and the percentage of the maximum sum (area) of the high heart rates is 3%, and thus the change in heart rate is not severe.

However, in the 40-minute section before the occurrence of IDH, the percentage of the maximum sum of the low heart rates and the percentage of the maximum sum of the high heart rates account for almost 50%, which may be predicted to be the occurrence of IDH.

In addition, the maximum value of the sum of each of high and low heart rates may be set to 6,000. This may be empirically set to generate an alarm when the systolic blood pressure decreases by 20 mmHg or more for 5 minutes or more during 40 minutes before the occurrence of IDH.

Accordingly, the processor 30 may determine that the blood pressure continuously decreases and generate an alarm when the sum of the heart rates of the average heart rate or higher or the sum of the heart rates of less than the average heart rate exceeds the set maximum value of 6,000 heart rates for 5 minutes or more on the basis of 5 minutes, which is the set cycle.

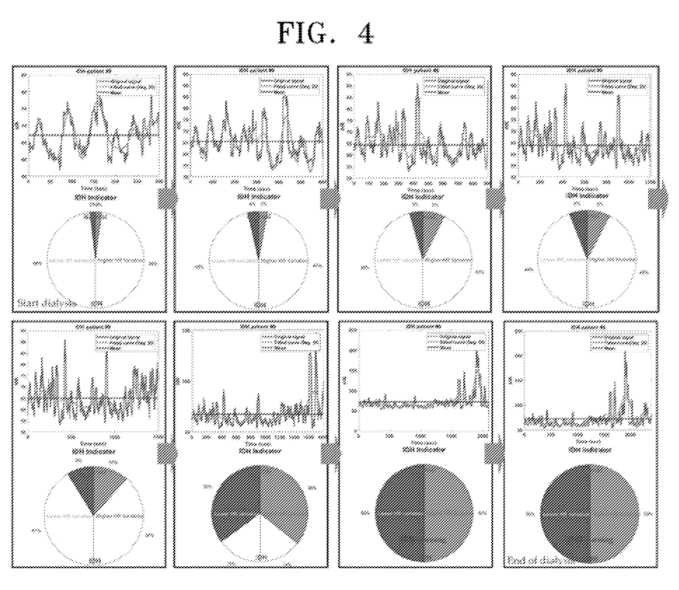

FIG. 4 is an exemplary view showing an IDH indicator of a hypotension patient by each time section during a set time in the apparatus for predicting IDH according to one embodiment of the present invention.

As shown in FIG. 4, the results of the IDH indicator from the initial 5 minutes to the last 40 minutes up to 5 minutes before the occurrence of IDH in a patient who was subjected to the occurrence of IDH can be shown.

From the initial 5 minutes to 25 minutes, the heart rate fluctuates up and down, but has no great fluctuation, and thus a change rate of the heart rate in the IDH indicator is not great. However, after 30 minutes has elapsed, it can be seen that, as the heart rate increases, the change rates of the high and low heart rates in the IDH indicator are great.

This is because a rapid increase in a heart rate (an increase in blood pressure) caused by the blood pressure compensatory mechanism occurs after the low heart rate section due to low blood pressure (although not severe) for a long time up to 30 minutes, and such a rapid increase in the heart rate is one of common precursor phenomena before the occurrence of IDH.

After the rapid increase in the heart rate due to the compensatory mechanism, a much lower hypotension state may occur, which can increase the probability of causing IDH.

Thereafter, as a long-term hypotension section and a rapid hypertension section appear in the 40-minute-long heart rate signals up to the time point of 5 minutes before the occurrence of IDH, the change rates of the high and low heart rates of the IDH indicator reach maximum values, which may be predicted to be IDH to output an alarm.

As described above, the apparatus for predicting IDH in a patient according to the embodiment of the present invention may predict the occurrence of IDH in a patient in real time before the occurrence of IDH through a rapid increase in a heart rate due to a blood pressure compensatory mechanism after a low heart rate section using a heart rate fitting curve and an average heart rate with respect to a heart rate signal of a dialysis patient.

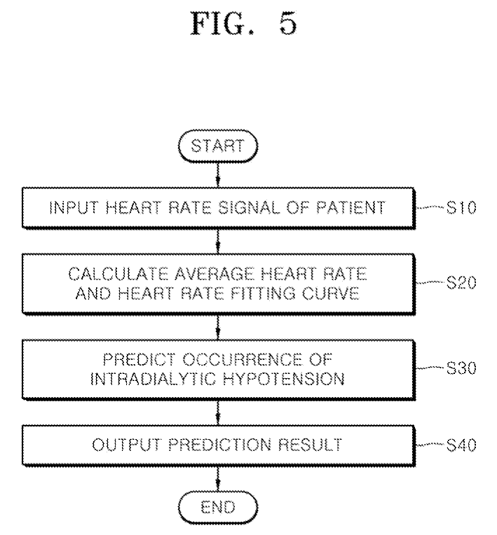

FIG. 5 is a flowchart for describing a method of predicting IDH in a patient according to one embodiment of the present invention.

As shown in FIG. 5, in the method of predicting IDH in a patient according to one embodiment of the present invention, first, the processor 30 executes the execution program stored in the memory 20 and then receives the heart rate signal of the patient measured by the measurement module 10 when the user selects measurement start through the input/output module 50 (S10).

In this case, the processor 30 may store the received heart rate signal in the database 40 for the set time, i.e., 40 minutes in the present embodiment.

Here, the measurement module 10 may include an electrocardiograph and measure and provide a heart rate at 1-second interval.

When receiving the heart rate signal of the patient in operation S10, the processor calculates the average heart rate and the heart rate fitting curve of heart rate signals for the set time at the set cycle interval (S20).

Here, the processor 30 may calculate the average heart rate and the heart rate fitting curve by fitting a multi-order polynomial on the heart rate signals.

In the present embodiment, the processor 30 may calculate the heart rate fitting curve fitted with the 20^{th}-order polynomial and identify the overall trend of the heart rate.

In operation S20, after calculating the average heart rate and the heart rate fitting curve of the heart rate signal, the processor 30 analyzes the change in heart rate based on the average heart rate and the heart rate fitting curve and predicts the occurrence of IDH (S30).

The processor 30 may analyze the change in heart rate based on the average heart rate and the heart rate fitting curve for the set time at the set cycle interval.

That is, the processor 30 may predict the occurrence of IDH when at least one of the percentage of the maximum area of the areas of the average heart rate or higher in the heart rate fitting curve and the percentage of the maximum area of the areas of less than the average heart rate in the heart rate fitting curve for the set time at the set cycle interval is the set value or more.

As shown in FIG. 3, in each heart rate signal, based on the average heart rate, the blue circle represents the area corresponding to the maximum sum of high heart rates, and the red circle represents the area corresponding to the maximum sum of low heart rates.

In this case, left and right sides of the IDH indicator that indicates a change in heart rate represent rates of the maximum sums of the low heart rates and the high heart rates, respectively, which can indirectly show incidence rates of low and high blood pressures.

That is, in the initial 5-minute (300 point) section, the percentage of the maximum sum (area) of the low heart rates is 5%, and the percentage of the maximum sum (area) of the high heart rates is 3%, and thus the change in heart rate is not severe.

However, in the 40-minute section before the occurrence of IDH, the percentage of the maximum sum of the low heart rates and the percentage of the maximum sum of the high heart rates account for almost 50%, which may be predicted to be the occurrence of IDH.

In addition, the maximum value of the sum of each of high and low heart rates may be set as 6,000 heart rates. This may be empirically set to generate an alarm when the systolic blood pressure decreases by 20 mmHg or more for 5 minutes or more during 40 minutes before the occurrence of IDH.

Accordingly, the processor 30 may determine that the blood pressure continuously decreases and generate an alarm when the sum of the heart rates of the average heart rate or higher or the sum of the heart rates of less than the average heart rate exceeds the set maximum value of 6,000 heart rates for 5 minutes or more on the basis of 5 minutes, which is the set cycle units.

After predicting the occurrence of IDH in operation S30, the processor 30 outputs the prediction result through the input/output module 50 (S40).

As described above, the apparatus for predicting IDH in a patient according to the embodiment of the present invention may predict the occurrence of IDH in a patient in real time before the occurrence of IDH through the rapid increase in a heart rate due to the blood pressure compensatory mechanism after the low heart rate section using the heart rate fitting curve and the average heart rate with respect to the heart rate signal of a dialysis patient.

An apparatus and method for predicting IDH in a patient according to an aspect of the present invention can predict the occurrence of IDH in real time before the occurrence of IDH through a rapid increase in a heart rate due to a blood pressure compensatory mechanism after a low heart rate section using a heart rate fitting curve and an average heart rate with respect to a heart rate signal of a dialysis patient.

Effects of the present invention are not limited to those described above, and other effects that are not specifically mentioned herein will be clearly understood by those skilled in the art based on the description of the present invention below.

Although the present invention has been described with reference to embodiments shown in the drawings, these are merely illustrative, and those skilled in the art will understand that various modifications and equivalent other embodiments are possible therefrom.

Accordingly, the technical scope of the present invention should be determined by the appended claims.

What is claimed is:

1. An apparatus for predicting intradialytic hypotension in a patient, comprising:
a measurement module configured to measure a heart rate of a patient;
a database; and
a processor operatively coupled to the measurement module and the database,
wherein the processor calculates an average heart rate and a heart rate fitting curve of heart rate signals of the patient measured by the measurement module for a set time, analyzes a change in heart rate based on the average heart rate and the heart rate fitting curve, and predicts occurrence of intradialytic hypotension.

2. The apparatus of claim 1, wherein the processor calculates the heart rate fitting curve by fitting a multi-order polynomial on the heart rate signals.

3. The apparatus of claim 1, wherein the processor analyzes the change in heart rate based on the average heart rate and the heart rate fitting curve for the set time at a set cycle interval.

4. The apparatus of claim 1, wherein the processor predicts the occurrence of intradialytic hypotension when at least one of a percentage of a maximum area among areas of the average heart rate or higher and a percentage of a maximum area among areas of less than the average heart rate in the heart rate fitting curve for the set time at a set cycle interval is a set value or more.

5. The apparatus of claim 1, wherein the processor generates an alarm when a sum of heart rates of the average heart rate or higher or a sum of heart rates of less than the average heart rate at each set cycle exceeds a set maximum value.

6. A method of predicting intradialytic hypotension in a patient, comprising:
receiving, by a processor, heart rate signals of a patient measured by a measurement module;
calculating, by the processor, an average heart rate and a heart rate fitting curve of the heart rate signals for a set time; and
analyzing, by the processor, a change in heart rate based on the average heart rate and the heart rate fitting curve and predicting occurrence of intradialytic hypotension.

7. The method of claim 6, wherein the calculating of the heart rate fitting curve includes calculating, by the processor, the heart rate fitting curve by fitting a multi-order polynomial on the heart rate signals.

8. The method of claim 6, wherein the analyzing of the change in heart rate and predicting of the occurrence of intradialytic hypotension includes analyzing, by the processor, the change in heart rate based on the average heart rate and the heart rate fitting curve for the set time at a set cycle interval.

9. The method of claim 6, wherein the analyzing of the change in heart rate and predicting of the occurrence of intradialytic hypotension includes predicting, by the processor, the occurrence of intradialytic hypotension when at least one of a percentage of a maximum area among areas of the average heart rate or higher and a percentage of a maximum area among areas of less than the average heart rate in the heart rate fitting curve for the set time at a set cycle interval is a set value or more.

10. The method of claim 6, wherein the analyzing of the change in heart rate and predicting of the occurrence of intradialytic hypotension includes generating, by the processor, an alarm when a sum of heart rates of the average heart rate or higher or a sum of heart rates of less than the average heart rate at each set cycle exceeds a set maximum value.
